(19) United States
(12) Patent Application Publication
(10) Pub. No.: US 2018/0185248 A1
(43) Pub. Date: Jul. 5, 2018

(54) GASTRIC PROBE

(71) Applicant: SIMEX MEDINZINTECHNIK GMBH, Deisslingen-Laufen (DE)

(72) Inventor: RAINER SIRING, DEISSLINGEN (DE)

(21) Appl. No.: 15/907,511

(22) Filed: Feb. 28, 2018

Related U.S. Application Data
(63) Continuation of application No. PCT/EP2016/069850, filed on Aug. 23, 2016.

(30) Foreign Application Priority Data
Aug. 28, 2015 (DE) 10 2015 216 469.1

(51) Int. Cl. A61J 15/00; A61M 1/00

(52) U.S. Cl. CPC A61J 15/0073; A61M 1/0086; A61J 15/0003

(57) ABSTRACT

A gastric probe, which can be inserted through the nose of a patient, includes a hose for nasoenteral feeding of the patient and/or for discharging gastric juice. An extraction tube is secured to the hose and has, at a distal end, an extraction section which includes a plurality of openings. The extraction section, when inserted, is placed in the pharynx of the patient in order to extract saliva from the pharynx.

### CROSS-REFERENCE TO RELATED APPLICATION

This application is a continuation, under 35 U.S.C. § 120, of copending International Application PCT/EP2016/069850, filed Aug. 23, 2016, which designated the United States; this application also claims the priority, under 35 U.S.C. § 119, of German Patent Application DE 10 2015 216 469.1, filed Aug. 28, 2015; the prior applications are herewith incorporated by reference in their entirety.

### BACKGROUND OF THE INVENTION

### Field of the Invention

The invention relates to a gastric probe which can be inserted through the nose of a patient, including a hose for nasoenteral feeding of the patient and/or for removal of gastric juices.

Such gastric probes are generally known. In order to artificial feed a patient, the gastric probe is inserted or pushed in through the nose and through the pharyngeal space and the esophagus into the stomach or even as far as the intestine. The feed hose of the gastric probe has two ends, namely a distal end which, in the inserted state, is positioned in the stomach or in the intestine, and a proximal end, which is positioned outside the patient and usually serves for the supply of food. Placement of a gastric probe is a routine procedure in hospitals and is also performed on stroke patients. It is also known from tests that, in the case of stroke patients, there is a high risk of pneumonia.

### SUMMARY OF THE INVENTION

It is accordingly an object of the invention to provide a gastric probe, which overcomes the hereinafore-mentioned disadvantages of the heretofore-known devices of this general type and which reduces the risk of pneumonia, particularly in stroke patients.

With the foregoing and other objects in view there is provided, in accordance with the invention, a gastric probe to be inserted through the nose of a patient, including a feed hose for nasoenteral feeding of the patient and/or for removal of gastric juice. A suction hose which, at a distal end, has a suction portion with a number of openings is additionally secured on the feed hose. In relation to the feed hose, the suction portion is disposed in such a way that, in an inserted state, it is placed in the pharynx, i.e. the pharyngeal space of the patient, in order to suction saliva or secretions from the pharynx.

The advantages afforded by the invention are in particular that the gastric probe is equipped with an additional suction lumen and thus with a possibility of suctioning in order to effectively prevent unwanted entry of saliva into the trachea. The saliva is thus advantageously removed through the suction portion before reaching the trachea. The suction hose is secured on the feed hose in such a way that, in the inserted state of the gastric probe, the suction portion of the suction hose is placed in the pharynx of the patient and in particular close to or even ahead of the larynx. Saliva is then suctioned particularly effectively before the branching into the trachea and esophagus. Overall, the risk of pneumonia in a patient intubated with a gastric probe is thus considerably reduced.

This embodiment is based on the consideration that stroke patients often experience difficulty swallowing, with a risk of aspiration, i.e. a risk of saliva and generally secretions and/or particles entering the trachea. These then pass through the trachea into the lungs and can there become the cause of the pneumonia. This is also based on the knowledge that swallowing difficulties in stroke patients are often undetected, since their diagnosis typically requires a specialist, who is often not available.

With the device proposed herein, it is thus ensured that the risk of pneumonia is reduced in stroke patients who are being artificially fed. An explicit diagnosis of dysphagia is therefore not required. The particular advantage of the gastric probe described herein is, among other things, that precautionary suctioning can take place as it were automatically, without the need for special diagnoses or for special further measures.

It is also particularly advantageous, on one hand, that no additional oral suctioning is needed and the oral cavity is accordingly kept free, and, on the other hand, that it is also possible to dispense with an intubation or a tracheotomy to avoid aspiration, i.e. to dispense with a surgical intervention.

In patients who are being ventilated, so-called tracheal cannulas are often used, as are known from International Publication WO 2011/054507 A1, corresponding to U.S. Pat. No. 9,463,297, for example. The tracheal cannula is inserted through a tracheal incision in the trachea. In order to prevent aspiration in tracheal cannulas of that kind, it is known to provide the tracheal cannula with suction and to apply a cuff to the tracheal cannula, which cuff ensures sealing, in such a way that no secretions can enter into the lungs.

With the gastric probe described herein, the artificial feeding and also the suctioning are effected in a particularly simple and compact way through the nose. In other words, nasoenteral nutrition is combined with nasopharyngeal suction by using the gastric probe.

The feed hose of the gastric probe serves primarily to supply food or also medicaments to the digestive tract. The feed hose is accordingly dimensioned for this purpose and has a distal end which, in the inserted state of the gastric probe, is positioned in the stomach or in the intestine and has an opening for the discharging of substances transported through the hose. Moreover, the feed hose has a proximal end which remains outside the patient even after insertion and serves primarily to supply food. In one variant, the feed hose has several proximal ends for supplying different substances or for performing additional functions, for example removal of gastric juice.

The suction hose is connected to the feed hose and secured thereto, for example affixed thereto, or is formed in one piece with the feed hose, for example by common extrusion. The suction hose in this case extends substantially in the same direction as the feed hose. Overall, this ensures a particularly safe positioning of the suction hose during insertion. Moreover, the suction hose and the feed hose each have an interior or also a lumen, wherein the two interiors are not directly connected to each other but separated from each other, in such a way that no exchange takes place between the suction hose and the feed hose.

For insertion, the gastric probe is pushed, starting with the distal end, into the pharyngeal space through the nose. The feed hose is then advanced onward past the larynx and through the esophagus into the stomach or into the intestine. By contrast, the suction hose is shorter than the feed hose and does not reach into the esophagus, and instead it terminates in the pharynx or just beyond, i.e. at most shortly behind the larynx or just in front. In this way, the suction portion, which is disposed at the distal end of the suction hose, is optimally positioned for suctioning in the pharynx. The lengths of the feed hose and of the suction hose and the securing position of the suction hose on the feed hose are therefore chosen in such a way that, in the inserted state, the distal end of the feed hose terminates at the intended position at the stomach and the distal end of the suction hose terminates above the larynx. Proceeding from a common starting point before or in the nose, the feed hose is therefore longer than the suction hose, for example by the length of the esophagus, minus any additional lengths in the digestive tract.

In cross section, the feed hose has in particular a cross-sectional area in the range of 1.7 to 22 mm². In the case of a circular cross section, this corresponds to a diameter in the range of approximately 1.5 to 5.3 mm. A diameter in the range of 2 to 4 mm is particularly preferred. In cross section, the suction hose has in particular a cross-sectional area in the range of approximately 0.25 to 20 mm². In the case of a circular cross section, this corresponds to a diameter in the range of approximately 0.6 to 5 mm.

The openings on the suction portion serve primarily to receive liquid, in particular saliva or secretions in the pharynx, i.e. the pharyngeal space. In a first variant, an opening is present at least at the end of the suction hose, i.e. on a front face at the distal end. In other words, the suction hose is open in the longitudinal direction.

Preferably, the openings are additionally, preferably alternatively, introduced in the radial direction into a wall of the suction hose. Generally, it is preferable for a large number of relatively small openings to be disposed on the suction portion, where relatively small is preferably understood to mean that a respective opening has a diameter in the range of approximately 0.1 to 4 mm, particularly preferably in the range of 1 to 3 mm. The openings are not necessarily circular, but for example oval. The diameter of the opening is then measured in particular in the direction of the greatest extent. In particular, the openings are limited to the suction portion. Therefore, no further openings are formed above the suction portion.

The openings in the wall of the suction hose are preferably distributed uniformly and also have identical diameters. This ensures homogeneous suctioning along the entire length of the suction hose.

In one suitable variant, the suction hose is formed by the gastric probe being configured in part with a double wall, and the openings are introduced into an outer wall. In other words, the gastric probe is double-walled on a certain portion toward the proximal end, wherein an inner wall separates the interior of the suction hose from the interior of the feed hose, and an outer wall delimits the interior of the suction hose from the environment. This integration of the suction hose results in a particularly compact gastric probe. In one embodiment, the suction hose then has no end face at the distal end.

Various configurations are conceivable in principle. In a first variant, the outer wall is as it were built onto the feed hose, in such a way that the suction hose protrudes radially from the feed hose and forms a thickening of the material. In a second variant, the suction hose is integrated in the interior of the feed hose, by the inner wall delimiting a subspace. In this embodiment, the gastric probe has in particular a constant diameter along the feed hose. The suction hose then leads to a partial tapering of the feed hose. In a third variant or in addition to one of the aforementioned variants, the suction hose is ring-shaped, in such a way that its interior surrounds the feed hose about the entire circumference. For this purpose, for example, the inner wall and the outer wall are disposed concentrically to each other. In each case, the openings on the suction portion are introduced only into the outer wall, in such a way that saliva, secretions and particles can only pass from outside into the suction hose.

In an alternative to the double-walled embodiment, the suction hose is disposed as a separate hose on the feed hose, in such a way that the two hoses, seen in cross section, have the shape of a figure eight lying on its side. The suction hose then in particular has a smaller diameter than the feed hose and the two loops of the figure eight are therefore not of the same size. The suction hose is connected cohesively to the feed hose, for example by adhesive bonding or welding. Alternatively, it is formed in one piece with the feed hose, for example in a common extrusion process in which the two hoses are formed with a common wall.

In an expedient embodiment, the openings are configured as a perforation of the suction hose. Such a perforation advantageously ensures a reduced loss or pressure and thus optimal suctioning. In the perforation, the diameter of each opening is preferably approximately 0.1 to 2 mm. Furthermore, the openings are distributed with a defined density on the suction portion, and the density is preferably approximately 1 to 4 openings per centimeter along the length of the suction hose. For example, 3 to 10 openings with a diameter of in each case approximately 1.2 mm are disposed on a suction portion with a length of 3 cm. The diameter of the suction hose is then 3 mm, for example.

The dimensions of the gastric probe are dependent in principle on the anatomy of the patient. In order to ensure optimal positioning of the suction region in the pharynx of the patient, the suction hose expediently has a length of approximately 20 to 25 cm. By contrast, the feed hose is much longer and is in particular approximately two to three times as long. In one variant, the feed hose is up to five times as long, in particular when it is used as a duodenal probe. This ensures that, in the inserted state, the distal end of the feed hose is positioned in the digestive tract, while the suction portion is positioned in the pharynx.

The suction portion preferably has a length in the range of 1 to 10 cm and is in this way then optimally adapted for the suctioning of saliva along the pharynx. A length of approximately 2.5 cm is particularly suitable.

In a particularly preferred embodiment, the suction portion can be cut to length and thus has in particular a variable length. Since the distal ends of the feed hose and of the suction hose are spaced apart from each other substantially by the length of the esophagus, but this length can differ from patient to patient, the suction portion that can be cut to length then makes it possible to optimally adjust a distance between the two distal ends as well. For this purpose, the suction portion, hence also the suction hose, is brought to a suitable length, i.e. shortened, and for this purpose cut, for example.

With regard to this cutting to length, a defined minimum length of the suction hose is expedient in order to ensure that a sufficient number of openings are still present, and that sufficient suction is guaranteed, even in the case of particularly small patients and a correspondingly great shortening of the suction portion.

The suction portion is advantageously releasable from the feed hose, as a result of which in particular a shortening of the suction portion is greatly simplified and accidental damage to the feed hose is advantageously prevented. The suction hose is preferably not secured along its entire length to the feed hose, but only in parts, in such a way that the suction portion can be folded away from the feed hose at least partially or even completely. However, in order to ensure an optimal positioning of the suction portion and in particular to avoid accidental folding away from the feed hose during insertion, the feed hose and the suction hose are suitably constructed in such a way that they fasten to each other, i.e. are connected releasably to each other, for example through adhesion forces due to suitably selected materials, surface treatments or suitable (adhesive) intermediate layers. Alternatively, this releasable connection is provided along the entire fastening length of the suction hose through suitable adhesion forces. Use is advantageously made of the fact that, in insertion through the nose, the feed hose and the suction hose are pressed against each other due to the generally small dimensions within the nose and then remain together on their further course.

Generally speaking, the parts of the gastric probe coming into contact with the patient are each composed of biocompatible materials, in particular biocompatible plastics.

In a particularly preferred embodiment, a number of length markings are applied to the suction hose at one end, which markings each indicate a distance to the other end of the suction hose. In a first variant, the length markings indicate a distance to the distal end, in such a way that, during insertion, the length marking can be read off to determine how far the distal end of the suction hose in particular and the gastric probe in general has already been pushed in.

In a preferred second variant, the length markings are applied to the suction portion, and the suction portion can be cut to length at the length markings, as a result of which the suction portion can then be prepared in a particularly simple manner. In this variant, the length markings each indicate in particular a distance to the proximal end of the suction hose. The latter is then cut to the appropriate length according to the specific anatomy of the particular patient.

Along the suction hose of the gastric probe, and in the region of the proximal end of the suction hose, it is advantageously possible to do without a cuff of the kind known in tracheal cannulas. A cuff is understood in particular as an inflatable bellows which is regularly used to provide positive sealing of hoses that are routed through hollow spaces. Due to the suctioning of saliva in the region of the pharynx, and in particular also of the larynx, it is possible in particular to dispense with closing the trachea by using a cuff, for which reason in particular the configuration of a cuff at the proximal side, i.e. above the larynx or before the latter, is preferably omitted.

By contrast, it is still conceivable to use a cuff in the esophagus, for example in order to prevent liquids ascending from the stomach. In an expedient embodiment of the gastric probe, a cuff is dispensed with entirely, i.e. the whole gastric probe is free of a cuff.

In particular at the proximal end, the suction hose suitably has an attachment piece for the attachment of a suction pump, as a result of which automatic suctioning in particular is permitted. The attachment piece is then a plug, for example, or a part of a coupling for connection to an attachment hose. A corresponding feeding system therefore includes, in addition to the gastric probe, also a suctioning device which is attachable and in particular also attached to the suction hose.

In order in particular to simplify the handling of the gastric probe, the suction hose branches off from the feed hose at the proximal end. In this way, the accesses through the proximal ends of the feed hose and of the suction hose can be positioned spatially independently of each other and are in this way more easily attachable to respective attachments. The gastric probe then accordingly has a branch at which the proximal end branches away from the feed hose and is then also in particular not connected to the feed hose.

Other features which are considered as characteristic for the invention are set forth in the appended claims.

Although the invention is illustrated and described herein as embodied in a gastric probe, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic, elevational view of a first variant of a gastric probe; and

FIG. 2 is an elevational view of a second variant of the gastric probe.

### DETAILED DESCRIPTION OF THE INVENTION

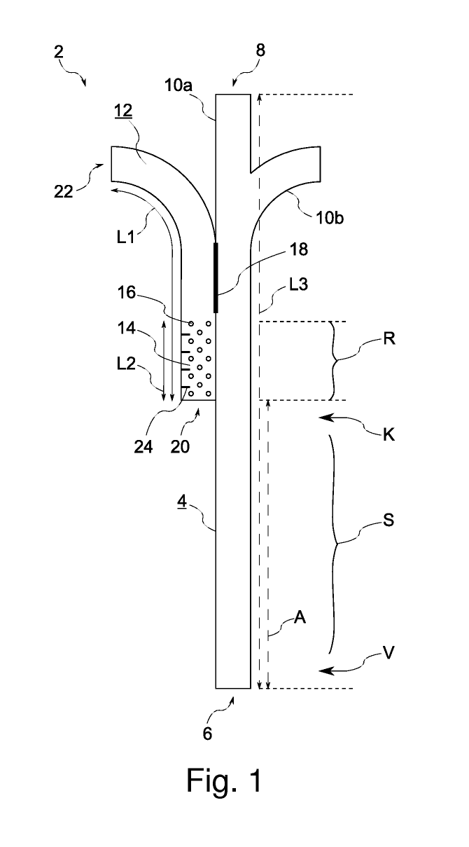

Referring now to the figures of the drawings in detail and first, particularly, to FIG. 1 thereof, there is seen a first variant of a gastric probe 2 which can be inserted through the nose of a patient for nasoenteral feeding of the patient. For this purpose, the gastric probe 2 has a feed hose 4 with a distal end 6 which, in the inserted state, is positioned in the stomach or in the intestine of the patient. Furthermore, the feed hose 4 has a proximal end 8 which, in the inserted state, remains outside the patient. Food, for example, is then supplied from the proximal end 8 for artificial feeding. In the embodiment shown herein, the proximal end 8 of the hose 4 has two accesses 10a, 10b, which can be used to supply different substances. For example, one access 10a is constructed for the attachment of a syringe containing food, and the other access 10b is constructed as an irrigation channel.

In addition to the feed hose 4 for nasoenteral feeding, the gastric probe 2 has a suction hose 12 for nasopharyngeal suctioning of saliva from the pharynx of the patient. For this purpose, the suction hose 12 has a suction portion 14 at which a number of openings 16 are introduced into the suction hose 12. The suction hose 12 and the suction portion 14 are additionally secured on the feed hose 4 at a portion 18 and are thus fixed in position relative to the latter. The suction hose 12 branches away from the feed hose 4 at the proximal end 8 of the feed hose 4.

In order to position the suction portion 14 in the pharynx in the inserted state of the gastric probe 2, the suction hose 12 is much shorter than the feed hose 4, so that the suction hose 12 in the inserted state terminates much higher up than the feed hose 4. In the inserted state, the distal end 6 of the gastric probe 2 then lies in the digestive tract of the patient, while the suction portion 14 is positioned in the pharynx of the patient. The suction hose 12 then has a distal end 20, which is at a distance A from the distal end 6 of the feed hose 4.

In order to illustrate the positioning of the gastric probe 2 in the inserted state, FIG. 1 indicates the respective positions of the pharynx R, the larynx K, the esophagus S and the digestive tract V of the patient. It can be clearly seen that the feed hose 4 extends into the digestive tract V, but the suction hose 12 only reaches as far as the larynx K at most, so that the suction portion 14 is routed through the pharynx R.

This effectively prevents aspiration, i.e. entry of saliva and general liquids and/or particles into the trachea which (although not shown) begins below the larynx K. A construction is generally also conceivable in which the suction portion 14 extends slightly beyond the larynx, but in particular by not more than approximately 1 cm, in order to provide suction within the entire pharynx R.

The suction hose 12 has a length L1 of approximately 20 to 25 cm, and the suction portion 14 at the distal end 20 of the suction hose 12 has a length L2 of approximately 5 to 10 cm, in particular 8 cm. By contrast, the feed hose 4 has a length L3, which corresponds to approximately two to three times the length L1 of the suction hose 12.

In the embodiment shown in FIG. 1, several length markings 24 are additionally applied to the suction portion 14, for example in the form of colored markings. These markings in this case indicate the distance to a proximal end 22 of the suction hose 12. In order now to space the two distal ends 6, 20 apart from each other in an optimal manner, the suction portion 14 in this case is constructed to be able to be cut to length, specifically in particular at the length markings 24. In order to then take the particular individual anatomical circumstances of a specific patient into account, the suction hose 12 is suitably cut to length prior to the insertion of the gastric probe 2, and an optimal distance A between the two distal ends 6, 20 is set.

In particular, to make it easier to cut to length, the suction hose 12 in FIG. 1 is partially releasable from the feed hose 4, specifically along the suction portion 14. The latter can then be easily folded away from or lifted away from the feed hose 4 and then cut to length, without damaging the feed hose 4 in so doing.

The suction hose 12 moreover has an attachment piece (not shown in any detail) at its proximal end 22, to which a suction pump can be attached to permit the suctioning.

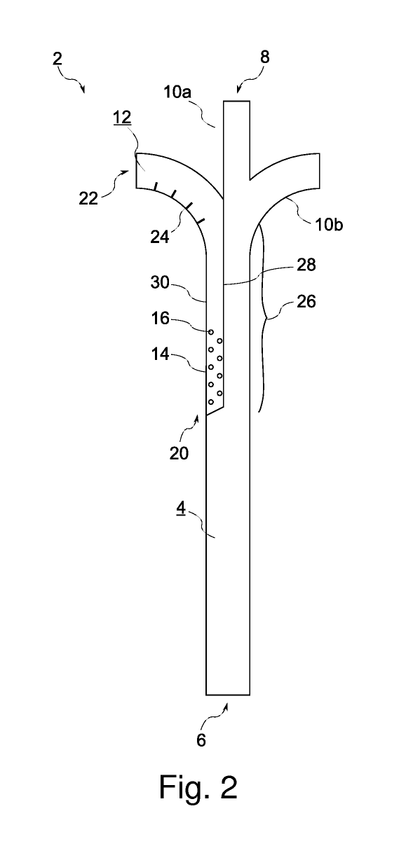

FIG. 2 shows a second variant of the gastric probe 2, in which the suction hose 12 is formed by a double-walled configuration of the feed hose 4. In this double-walled configuration, the feed hose 4 has a double-walled portion 26 at which an inner wall 28 separates the feed hose 4 from the suction hose 12. An outer wall 30 of the double-walled portion 26 then forms a delimitation of the suction hose 12 from the environment. The openings 16 are then also disposed on this outer wall 30.

In the illustrative embodiment shown in FIG. 2, the proximal end 22 of the suction hose 12 is additionally provided with length markings 24 which indicate the distance of the respective length marking 24 to the distal end 20 of the suction hose 12. In this way, it is then possible, when pushing the gastric probe 2 into the patient, to read off the length markings 24 so as to determine how far the gastric probe 2 has already been advanced.

The two illustrated variants of the gastric probe 2 are each free of a cuff. The latter is not required by virtue of the special construction, firstly because the positioning of the suction portion 14 in the pharynx R already greatly reduces the danger of aspiration. Closure of the pharynx R or of the esophagus S by using a cuff does not therefore take place. However, in a non-illustrated alternative, a cuff is applied to the feed hose 4 in the region of the esophagus S.

The following is a summary list of reference numerals and the corresponding structure used in the above description of the invention:
2 gastric probe
4 feed hose
6 distal end of the feed hose
8 proximal end of the feed hose
10a, 10b access
12 suction hose
14 suction portion
16 opening
18 portion
20 distal end of the suction hose
22 proximal end of the suction hose
24 length marking
26 double-walled portion
28 inner wall
30 outer wall
A distance
R pharynx
K larynx
S esophagus
V digestive tract
L1, L2, L3 length

1. A gastric probe to be inserted through the nose of a patient, the gastric probe comprising:
a feed hose for at least one of nasoenteral feeding of the patient or removal of gastric juice from the patient; and
a suction hose secured to said feed hose, said suction hose having a distal end and a suction portion disposed at said distal end;
said suction portion having a plurality of openings formed therein and said suction portion being placed in the pharynx of the patient in an inserted state in order to suction saliva from the pharynx.

2. The gastric probe according to claim 1, which further comprises a double-wall partial portion forming said suction hose and having an outer wall with said openings formed therein.

3. The gastric probe according to claim 1, wherein said openings are a perforation formed in said suction hose.

4. The gastric probe according to claim 1, wherein said suction hose has a length of approximately 20 to 25 cm.

5. The gastric probe according to claim 1, wherein said suction portion has a length in a range of 1 to 10 cm.

6. The gastric probe according to claim 1, wherein said suction portion has a length of approximately 2.5 cm.

7. The gastric probe according to claim 1, wherein said suction portion is configured to be cut to length.

8. The gastric probe according to claim 1, wherein said suction portion is releasable from said feed hose.

9. The gastric probe according to claim 1, wherein said suction hose has two ends and a plurality of length markings applied at one of said ends, each of said length markings indicating a respective distance from the other of said ends.

10. The gastric probe according to claim 9, wherein said length markings are applied to said suction portion, and said suction portion is configured to be cut to length at said length markings.

11. The gastric probe according to claim 1, wherein said suction hose has a proximal end, and said suction hose is free of a cuff along said suction hose and in a region of said proximal end.

12. The gastric probe according to claim 1, wherein said suction hose is configured to receive an attachment piece for attachment of a suction pump.

13. The gastric probe according to claim 1, wherein said feed hose has a proximal end, and said suction hose branches off from said feed hose at said proximal end.
